(19) United States
(12) Patent Application Publication
(10) Pub. No.: US 2003/0199819 A1
(43) Pub. Date: Oct. 23, 2003

(54) Filter wire system

(76) Inventor: Robert C. Beck, St. Paul, MN (US)

Correspondence Address: Beck & Tysver, P.L.L.C., Suite 100, 2900 Thomas Avenue S., Minneapolis, MN, 55416, US

(21) Appl. No.: 10/145,674

(22) Filed: May 14, 2002

Related U.S. Application Data
(60) Provisional application No. 60/373,117, filed on Apr. 17, 2002.

(51) Int. Cl. A61M 29/00

(52) U.S. Cl. 604/96.01; 606/200; 604/101.04

(57) ABSTRACT

A therapeutic interventional device such as a balloon catheter is provided with a nozzle to induce a retrograde flow in the vessel by injecting fluid through the nozzle into the vessel. The retrograde flow can be used to clear debris from a distal protection device such as a filter or balloon and may additionally be used to clear the vessel of clot prior to the intervention.

### CROSS REFERENCES

The present invention claims the benefit of co-pending application 10/050,978 filed Jan. 18, 2002, entitled Fluidic Interventional Device and Method of Distal Protection, which is incorporated by reference herein in its entirety.

The present application claims the benefit of provisional application 60/373,117 filed Apr. 17, 2002, entitled Filter Wire incorporated by reference in its entirety herein.

### BACKGROUND OF THE INVENTION

It is now widely recognized that cardiac interventions such as angioplasty can release an extraordinary amount of debris. If this debris flows downstream, it can clog vessels and propagate a cascade of injury. Although debris collection for the coronary arteries has been proposed, the primary application for “distal protection devices” is in saphenous vein graft interventions where occlusive material is friable and extensive, and in carotid interventions where the release of even small amounts of debris can lead to stroke or blindness and other neurological disorders.

The two dominant forms of distal protection device under investigation today include the Percusurge guard wire, which is a elastomeric occlusion balloon on a wire which is used to traverse a stenotic lesion and is inflated to block flow. A cardiovascular intervention such as stent placement, angioplasty, or artherectomy or the like takes place behind the occlusion balloon and is typically delivered over the guide wire portion of the balloon system. Although such systems have been proven safe and effective and have been released for marketing, there are continuing issues of “halo” and balloon shadow. It appears from clinical investigation that the occlusive balloon itself moves slightly in the vessel trapping debris between the balloon and the blood vessel. On the distal or downstream side of the device, blood stagnates around the outer periphery of the balloon and in the instance of a long intervention or an unheprinized patient this adherent material may form a ring or halo and be sloughed off as the occlusion balloon is deflated. Although such balloon-based systems achieve 100 percent occlusion of the vessel during the intervention, they are unable to extract 100 percent of the released debris either because the debris is trapped by the balloon or formed behind the balloon. In these instances, no amount of straight aspiration or irrigation followed by aspiration will remove the debris. The system taught by the present application permits 100 percent removal of occlusive material with the obvious patient benefit.

The alternative filter wire technology places a net or filter mesh distal across the lesion and material “created” or released during the intervention behind or proximal of the filter wire is collected in the filter wire basket. The typical filter wire has an approximately conical shape like a butterfly net and has sufficient volume to trap a relatively large amount of debris. However, there are instances where the quantity of debris or the quality of debris created during the intervention overwhelms the collection capacity of the filter wire and the filter wire itself becomes a total occlusion preventing the profusion of oxygenated blood to distal tissues. It is possible that the amount of debris is so large that the filter wire cannot be retrieved. The present invention permits the filter wire to be “emptied” peri-operatively which allows both profusion and retrieval.

### BRIEF DESCRIPTION OF THE DRAWINGS

FIG. 1 is a schematic diagram of a medical device in a vessel;

FIG. 2 is a schematic diagram of a medical device in a vessel;

FIG. 3 is a schematic diagram of a medical device in a vessel;

FIG. 4 is a schematic diagram of a medical device in a vessel;

FIG. 5 is a schematic diagram of a medical device in a vessel;

FIG. 6 is a schematic diagram of a medical device in a vessel;

FIG. 7 is a schematic diagram of a medical device in a vessel;

FIG. 8 is a schematic diagram of a medical device in a vessel;

FIG. 9 is a schematic diagram of a medical device in use in a vessel with a collection bag coupled to a guiding sheath.

### DETAILED DESCRIPTION

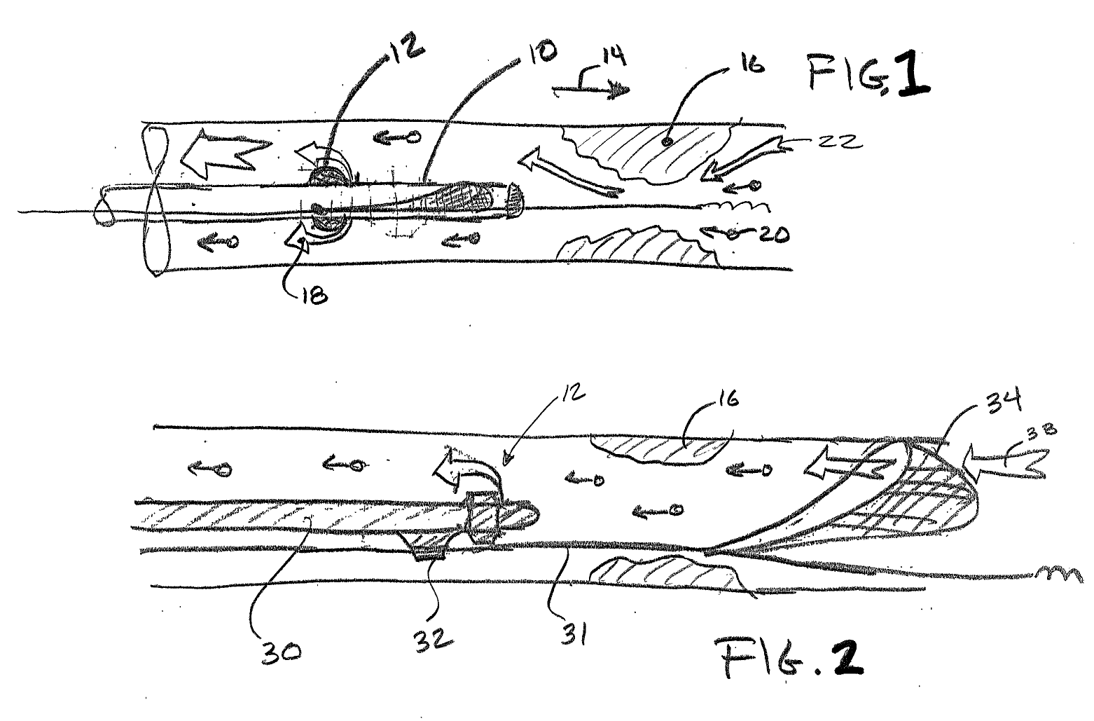

FIG. 1 shows a fluidic extraction nozzle 12 embodying the Coanda effect on a filter wire sheath 10. In use the filter wire sheath 10 is advanced antegrade as indicated by arrow 14 toward the lesion 16. With the extraction section 12 activated with heperinized saline or diluted contrast agent a flow is induced in the retrograde direction by primary jet 18 emerging from the extraction section 12. Debris released by the initial crossing of the lesion 16 is propelled in the retrograde direction as indicated by particle and motion arrow 20. These particles will be carried by the blood flow indicated by flow arrow 22. These particles will be collected in bag 810 seen in FIG. 9

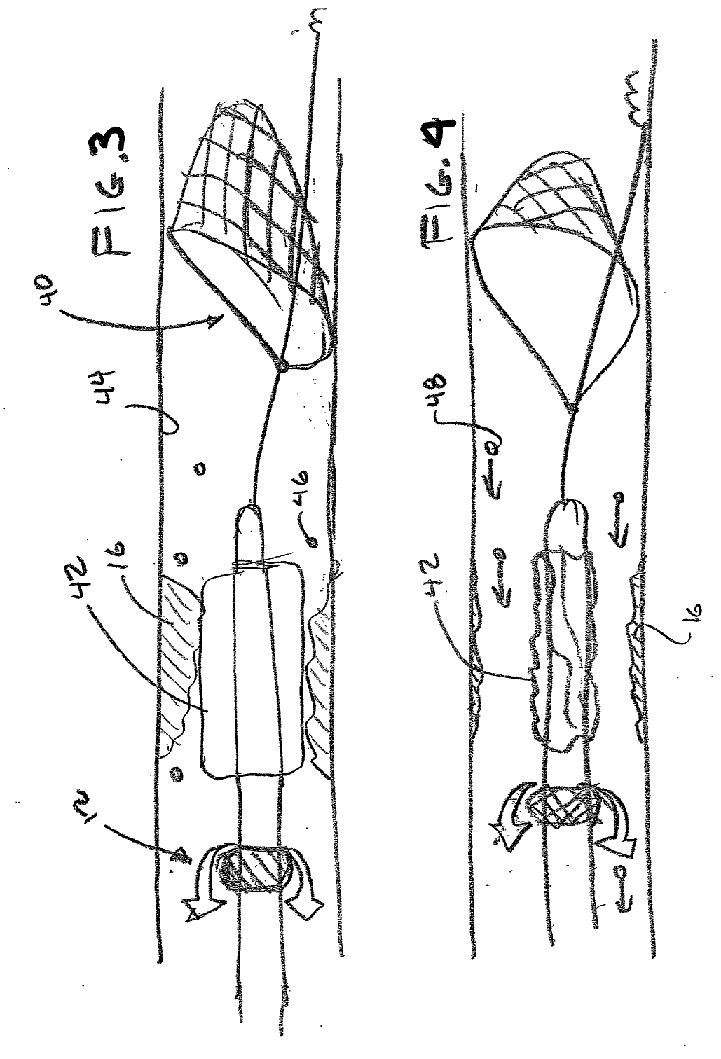

FIG. 2 shows a stand-alone extraction catheter 30 carried by a rapid exchange lumen 32 on the guide wire shaft 31 of a filter wire device. In this embodiment the extraction section 12 causes a pressure difference across the filter wire basket 34. The blood flows retrograde through the basket as indicated by arrow 38. In this embodiment he retrograde flow is used to “empty” the basket. This allows the clinician to liberate and collect large quantities of debris without concern. The filter will not get too full to remove. The debris will be in the bag 810 (FIG. 9).

FIG. 3 shows a filter wire 40 positioned to collect debris liberated by the angioplasty balloon 42. It is important to note that while the therapy balloon 42 is inflated there is essentially no flow in the vessel 44. The particulate typified by particle 46 is stagnant and not moving very far or very fast. If the extraction section 12 is turned on during the balloon inflation there will be a pressure difference created across the lesion 16.

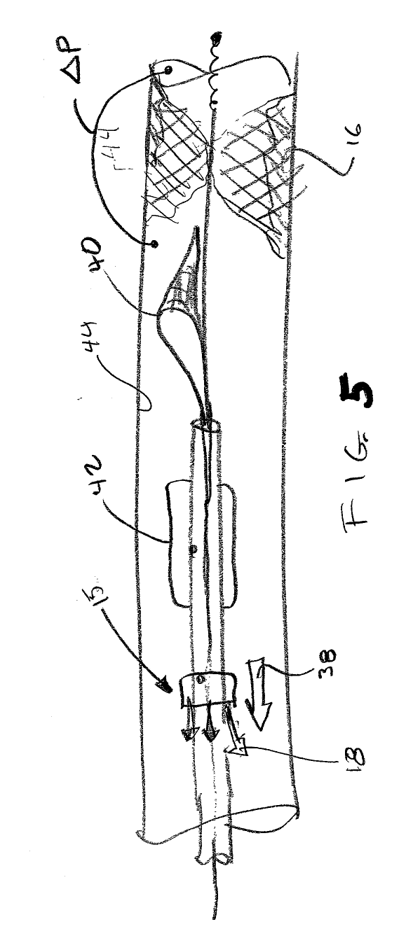

When the balloon is deflated as seen in FIG. 4 the particulate moves retrograde as typified by particle 48. In this instance the filter wire 40 acts as a safety net to capture debris in the unlikely event that the are not captured by retrograde flow.

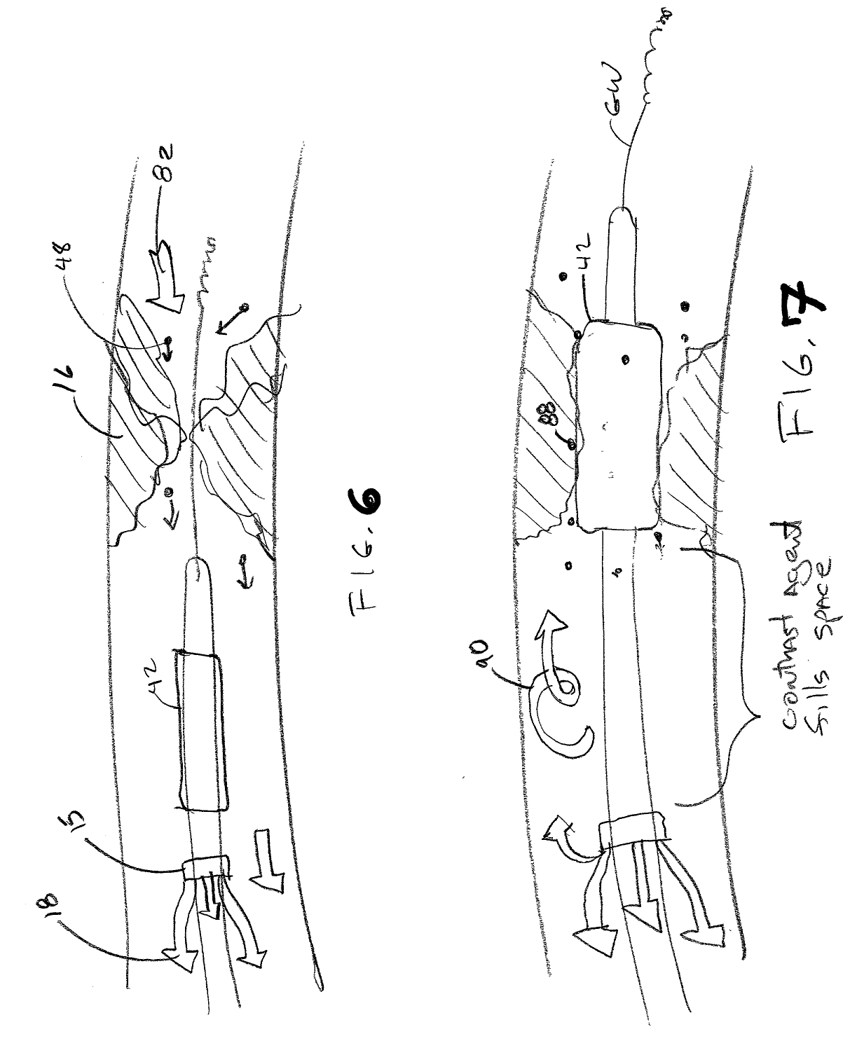

FIG. 5 shows the system of FIG. 1 further including a therapy balloon 42 added to the delivery sheath 10. This version uses an alternate design extraction section with a wall angle of about zero and a jet angle approaching 180 degrees. In this figure a pressure difference is created across the stenotic lesion 16 by the fluid ejected from extraction section 12. The filter wire 40 is shown partly deployed to show the construction of the sheath.

Turning to FIGS. 6 and 7 and 8 it is quite possible that effective distal protection of vessels can take place without the use of either filter or balloon occlusion devices as follows:

FIG. 6 shows a conventional guidewire 80 traversing a lesion 16. The extraction section 15 is injecting fluid 18 which may be dilute contrast agent or heprinized saline. As the lesion 16 is crossed the blood flow 82 induced by the retrograde flow 18 drags particles like particle 84 in the retrograde direction.

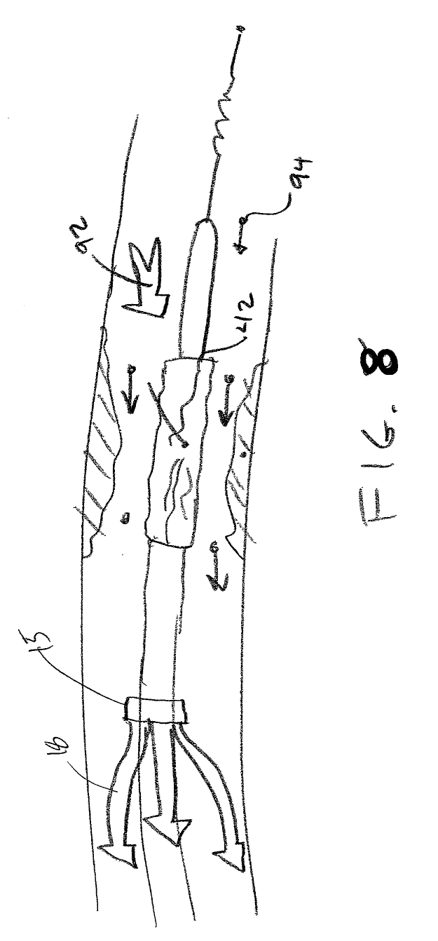

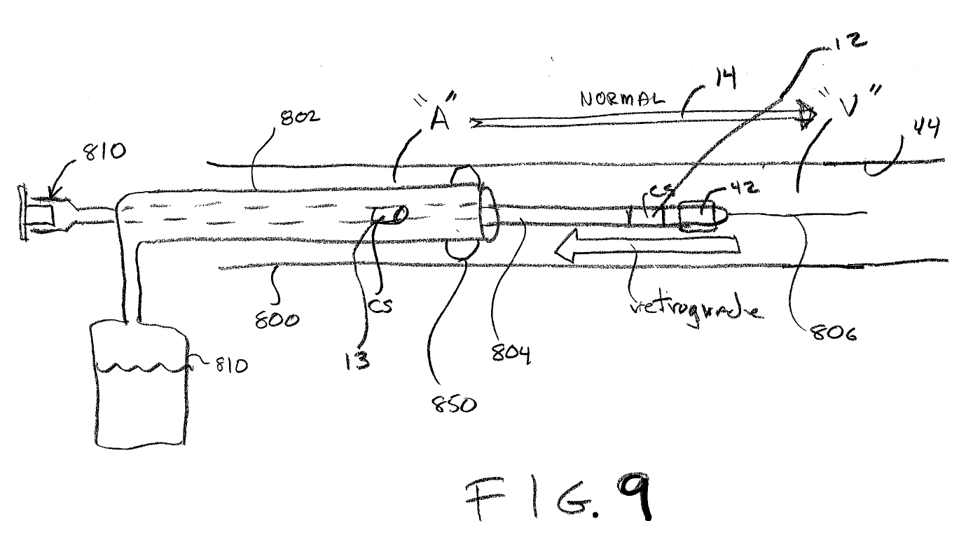

FIG. 7 shows the therapy balloon 42 pushed across the lesion 16 and inflated. The author believes that the bulk of the particles created are created by balloon expansion. However the balloon 42 now occludes the vessel and the particles like particle 88 is motionless since there is no blood flow. The extraction section continues to pump but the retrograde flow stops and the contrast agent mixes with the blood and displaces it through a serial dilution process indicated by arrow 90. The space behind the balloon fills with contrast agent and the doctor has a visual confirmation that the therapy balloon has occluded the vessel. It is important to note that the pressure gradient across the therapy balloon will induce retrograde flow as soon as the balloon is even slightly deflated as illustrated in FIG. 8.

FIG. 8 shows the therapy balloon 42 in a collapsing condition which opens the vessel 44 permitting full retrograde flow as indicated by arrow 92. Even particles that have migrated in the distal direction are captured and carried out to bag 802 by the injected flow 18. The physician will see the contrast agent swept from view in the retrograde direction confirming adequate particulate capture. Doctors will think this is really cool and the patients get a great benefit at a very low cost.

In the figures two different geometries of extraction sections are taught. Although these may be readily substituted for each other throughout the figures, they differ in some regards. The section illustrated generally as 12 consists of a set of radial projecting apertures which introduce fluid at a jet angle of approximately 90 degrees with the center axis of the catheter. A nubbin is located adjacent the slits and this nubbin guides the flow into the retrograde path. Such devices are further described elsewhere in my published patents and appear to be particularly useful when one desires to use contrast agent as the injectate to drive the extraction section. In these instances the volume between the aperture and the occlusion device which may be a therapy balloon or a distal occlusion balloon fills up quickly with contrast agent permitting the visualization of the lesion as well as the position of the occlusion element. If the occlusion element is deflated, then the contrast agent is swept from the system through the retrograde pumping action of the extraction section providing a visual confirmation fluroscopically of the extraction of debris. This is particularly helpful for balloon-based interventions where the occlusions prevent the introduction of contrast agent using conventional techniques. Physicians like the additional flexibility associated with being able to see what they're doing wherever they are in the course of the procedure. The nubbin of the extraction section is positioned with a wall angle of approximately 0 degrees that as the jet approaches the nubbin surface on a tangent. Other wall angles can be utilized and in particular a wall angle of about 45 degrees seems to promote a rapid filling of the treatment volume when injected with fluid.

An alternative geometry for the Coanda extraction section is set forth on FIGS. 6,7 and 8 which show a cuff or cup over one or more apertures. In this construction injectate fluid enters the cuff from a lumen in the catheter body and squirts out the back. The jet angle is approximately 180 degrees while the wall angle is nearly 0 degrees as the jet attaches to the catheter shaft and flows in the retrograde direction. This geometry establishes a good pressure recovery for the energy within the jet and creates a perceptible pressure difference across the therapy balloon or the occlusion balloon. The mixing process is not as vigorous with this geometry and if it is used against a total occlusion, the treatment volume takes substantially longer to fill with contrast agent. It is likely that the optimal geometry is intermediate between a Coanda extraction section having a jet angle between 90 and 180degrees and a wall angle of between 0 and 45 degrees.

FIG. 9 shows the overall context of the system where the patient's blood vessel 800 carries an interventional guide sheath 802 which in turn delivers an extraction catheter 804. The extraction catheter may be delivered over a guide wire 806, or it may be delivered without the benefit of a guide wire and lie loose in the extraction sheath 802. Injectate is forced into the catheter 804 through an injector 810 which will typically be an angiographic power injector, although in certain versions hand injection may be useful as well. The extraction sheath and guide catheter sheath 802 together form a collection system which will terminate in a collection bag 810 placed bedside next to the patient. In general if this bag is placed below the patient, the patient will bleed into the bag through arterial pressure and gravitational siphon. If the bag is placed above the patient, debris and the like in the bag would be reintroduced into the patient. In most instances the Coanda extraction section on the extraction catheter 804 will produce an output pressure of several inches of water which will be sufficient to take material in the antegrade flow induced by the Coanda extraction section into the guide catheter 802 and deposit the material in the collection bag 810 where it can be examined and filtered to determine the content, nature and amount of debris recovered.

To assist entry of debris into the open mouth of the guide catheter 802, there are three solutions. First a balloon 850 may be used to seal the space between the vessel wall 44 and the catheter body. Next a supplemental pumping station may be placed in the lumen of the device 802. The extraction section 13 may be powered at the same time as the more distal extraction section 12. The two extractions sections 13 and 12 may be operated at different times and for different duration. A third solution is the application of suction from a syringe or the like to the lumen of the sheath device 802. Any of these solutions may used separately or they may be combined in any permutation.

What is claimed

1. A method for extracting debris from a vessel having a lesion comprising the steps of:
placing a therapy catheter in contact with a lesion;
inflating the therapy balloon to treat the lesion producing debris;
injecting fluid into a extraction section creating a pressure gradient across the therapy balloon while it is inflated;
deflating the therapy balloon while injecting fluid to promote a retrograde flow across the surface of the therapy balloon entraining, capturing and moving debris in the retrograde direction.

2. The method of claim 1 further including the step of extracting said debris from a location proximal of said extraction section with a tube.

3. The method of claim 1 further comprising an initial step of traversing a treatable lesion with an occlusion device and deploying the occlusion device distal of said therapy balloon.

4. The method of claim 2 wherein said distal occlusion device is a filter.

5. The method of claim 2 wherein said distal occlusion device is an inflatable balloon.
